(19) United States
(12) Patent Application Publication
(10) Pub. No.: US 2014/0308749 A1
(43) Pub. Date: Oct. 16, 2014

(54) QUANTITATION OF LACTOFERRIN IN INFANT FORMULAS BY ELECTROPHORESIS USING IR FLUORESCENCE IMAGING

(71) Applicants: Cuiyi Yi, Audubon, PA (US); Ming Gao, Audobon, PA (US); Ping Feng, Audubon, PA (US); Anita Burgher, King of Prussia, PA (US)

(72) Inventors: Cuiyi Yi, Audubon, PA (US); Ming Gao, Audobon, PA (US); Ping Feng, Audubon, PA (US); Anita Burgher, King of Prussia, PA (US)

(73) Assignee: NESTEC S.A., Vevey (CH)

(21) Appl. No.: 13/992,188

(22) PCT Filed: Dec. 5, 2011
(86) PCT No.: PCT/IB2011/055470

§ 371 Date: Jul. 26, 2013

Related U.S. Application Data
(60) Provisional application No. 61/421,278, filed on Dec. 9, 2010.

(51) Int. Cl. G01N 33/68

(52) U.S. Cl. CPC G01N 33/68
USPC 436/20

(57) ABSTRACT

A method of quantitatively measuring the total lactoferrin content of an infant formula using electrophoresis and infrared fluorescence detection.

### BACKGROUND OF THE INVENTION

Lactoferrin is an iron-binding glycoprotein which is a natural component of milk, including human milk and bovine milk. Lactoferrin is believed to have antimicrobial, anti-inflammatory and immunomodulating activity, and other benefits.

Lactoferrin may occur in infant formula either because the formula includes milk components that contain lactoferrin or because lactoferrin has been added to the formula. To ensure that the infant consuming the formula receives an appropriate amount of lactoferrin, it is important to be able to accurately measure the amount of lactoferrin in infant formula.

One challenge in measuring the lactoferrin content of infant formula is presented by the heat treatment that the formula may undergo. Heat treatment causes proteins to denature and aggregate; disulfide bonds may form between protein molecules, bonding them together and making analysis difficult. For example, lactoferrin may bond to other proteins and form aggregates that will not permit the lactoferrin to be detected by standard protein analytical methods. As a result, analysis of the lactoferrin content of a heat treated infant formula would be expected to yield a lower result than for a non-heat treated infant formula, and not provide a result that reflects the total lactoferrin content.

One method for analyzing proteins so that both natured and denatured proteins will be measured is to use sodium dodecylsulfate polyacrylamide gel electrophoresis (SDS-PAGE) under reducing conditions. SDS-PAGE under non-reducing conditions cannot measure lactoferrin that is covalently bonded to other proteins by disulfide bonds to form aggregates. To break the disulfide bonds and disrupt the aggregates into individual proteins, the SDS-PAGE must be conducted under reducing conditions. This was demonstrated by G. Brisson, et al., Effect of Iron Saturation on the Recovery of Lactoferrin in Rennet Whey Coming from Heat-Treated Skim Milk, J. Dairy Sci. 90, 2655-2664 (2007). However, Brisson, et al., did not demonstrate a highly precise method for quantitatively measuring the amount of lactoferrin in the bands of the electrophoresis gel.

Coomassie blue is commonly used to stain gels. This dye makes the proteins visible so that the amount of protein may be measured semi-quantitatively by densiometry. Coomassie blue also presents the opportunity to use a more precise method of measurement than densitometry, because the dye fluoresces in the near infrared part of the electromagnetic spectrum, and quantitative measurements may be made using IR fluorescence detection, as demonstrated by S. Luo, et al., Quantitation of protein on gels and blots by infrared fluorescence of Coomassie blue and Fast Green, Anal. Biochem., 350, 233=238 (2006). The usefulness of this method for detecting proteins on gels was also demonstrated by L. R. Harris, et al., Assessing Detection Methods for Gel-Based Proteomic Analyses, J. Proteome Res., 6, 1418-1425 (2007).

There is a need to develop a reliable and accurate method for the precise quantitative measurement of total lactoferrin in an infant formula matrix

### SUMMARY OF THE INVENTION

The present invention provides a method of quantitatively measuring the total lactoferrin content of an infant formula. In this method, standard solutions having different known concentrations of lactoferrin are prepared and independently combined with a reducing sample buffer. The resulting solutions are loaded onto the same sodium dodecylsulfate polyacrylamide gel, heated for about 7-9 minutes at about 90-100° C., and then subjected to electrophoresis using substantially the same power level and time period for each solution. Following electrophoresis, the gel is stained with a gel stain that fluoresces in the infrared, and then destained, leaving on the gel only the stain that has bound to the lactoferrin in the standard solution. An infrared fluorescence imaging system is then used to quantitatively measure, at a suitable wavelength, the infrared fluorescence intensity of the stain for each standard solution to establish the relationship between measured infrared fluorescence and total lactoferrin.

Essentially the same procedure is used to analyze an infant formula containing an unknown amount of lactoferrin. The measured infrared fluorescence intensity of the stain on the gel containing the infant formula sample is used to determine the amount of lactoferrin in the infant formula sample by reference to the relationship between fluorescence and lactoferrin amount that was established using the standard solutions. Knowledge of the total amount of lactoferrin in the infant formula sample and the quantity of infant formula analyzed allows calculation of the lactoferrin concentration in the infant formula.

### DETAILED DESCRIPTION OF THE INVENTION

In the method of this invention, lactoferrin in an infant formula is measured by using sodium dodecylsulfate polyacrylamide gel electrophoresis under reducing conditions to separate lactoferrin molecules from other compounds in the sample being analyzed, and infrared fluorescence detection to obtain a quantitative result for the lactoferrin content of the sample.

The term “infant formula” as used herein refers to a nutritional formulation (either in the form of a liquid or in the form of a dry powder that may be reconstituted to form a liquid infant formula upon addition of water) that provides complete nutrition for an infant and may be used as a substitute for human milk in feeding an infant. Such formulae are well-known in the art.

Typically, an infant formula in a ready-to-consume liquid form provides 60-70 Kcal/100 ml. Infant formula typically comprises, per 100 Kcal: 1.8-4.5 g protein; 3.3-6.0 g fat (lipids); 300-1200 mg linoleic acid; 9-14 g carbohydrates selected from the group consisting of lactose, sucrose, glucose, glucose syrup, starch, malto-dextrins and maltose, and combinations thereof; and essential vitamins and minerals. Lactose may be the pre-dominant carbohydrate in an infant formula. A liquid infant formula may contain about 67 Kcal/100 ml. Infant formula may comprise about 1.8-3.3 g protein per 100 Kcal, for example, about 1.8-1.1 g protein per 100 Kcal.

An infant formula may also comprise nucleotides selected from cytidine 5′-monophosphate (CMP), uridine 5′-monophosphate (UMP), adenosine 5′-monophosphate (AMP), guanosine 5′-monophosphate (GMP) and inosine 5′-monophosphate (IMP), and mixtures thereof. Infant formula may also comprise lutein, zeaxanthin, fructo-oligosaccharides, galacto-oligosaccharides, sialyl-lactose, and/or fucosyl-lactose. Long chain polyunsaturated fatty acids, such as docosahexaenoic acid (DHA) and arachidonic acid (AA) may be included in infant formula. Infant formula may also include amino acids. Infant formula may also include other ingredients well-known in the art.

One gel stain well-known in the art which is useful in the practice of this invention is SimplyBlue™ SafeStain, a ready-to-use, proprietary Coomassie® G-250 stain that is commercially available from Invitrogen Corporation (Carlsbad, Calif., USA). When a gel containing protein (for example, lactoferrin) is treated with this gel stain, and the excess stain is washed from the gel, only the stain that is bound to the protein remains on the gel. Therefore the amount of stain remaining corresponds to the amount of protein.

Coomassie blue stain fluoresces in the near infrared at wavelengths of about 670-800 nm. The 680-700 nm range is suitable for measuring the fluorescence of Coomassie blue stained proteins. An infrared fluorescence scanner set to detect fluorescence at these wavelengths may be used to quantitatively measure the amount of Coomassie blue on a gel, and therefore to determine the amount of protein that binds the Coomassie blue. In one embodiment, the fluorescence intensity is measured at about 700 nm. In another embodiment, the fluorescence intensity is measured at about 680 nm.

In one embodiment, the invention is a method of quantitatively measuring the total lactoferrin content of an infant formula by: preparing a solution of the infant formula and combining it with a sample reducing agent to produce an infant formula sample solution; heating the infant formula sample solution at about 90-100° C. for about 7-9 minutes, for example at about 95° C. for about 8 minutes; loading the infant formula sample solution onto a sodium dodecylsulfate polyacrylamide gel to obtain a loaded gel; performing electrophoresis on the loaded gel; staining the gel with a Coomassie blue stain and then destain the gel, leaving on the gel only the stain that has bound to the lactoferrin; and, using an infrared fluorescence imaging system to quantitatively measure the infrared fluorescence intensity at a suitable wavelength of the stain on the gel. This intensity may be compared to the infrared fluorescence intensity at the same wavelength of standard solutions in the same gel having known concentrations of lactoferrin to determine the amount of lactoferrin in the infant formula. Those skilled in the art will readily understand how to use such data to calculate the total amount of lactoferrin, and the concentration of lactoferrin, in the infant formula sample. Those skilled in the art will also be able to readily determine a suitable wavelength without undue experimentation. For example, suitable wavelengths in the practice of this invention include, but are not necessarily limited to, those within the ranges of 670-800 nm, 670-700 nm, and 680-700 nm, such as 680 nm, 700 nm, 780 nm and 800 nm.

In one embodiment of this invention, the infrared fluorescence intensity of standard lactoferrin solutions is plotted versus lactoferrin content or concentration to provide a standard curve. The infrared fluorescence intensity of an infant formula of unknown lactoferrin concentration is compared to this curve to determine the lactoferrin content or concentration of the unknown sample. In one embodiment, the curve is substantially linear.

In infrared fluorescence spectroscopy, the material to be analyzed is irradiated with infrared radiation at one wavelength (the excitation wavelength) and the fluorescence at a longer wavelength (the fluorescence wavelength) is measured by the instrument. For example, in the detection of lactoferrin stained with Coomassie blue the material may be irradiated at about 550 nm while fluorescence may be measured at, for example, a wavelength in the range of about 680-800 nm. The wavelength of excitation is not limited to one particular wavelength, but may be any wavelength that produces excitation and fluorescence; for example, the excitation wavelength may be in the range of about 450-550 nm. Any excitation wavelength that will produce the desired fluorescence may be used, and those skilled in the art will readily be able to determine suitable excitation wavelengths. The fluorescence wavelength also may vary, since the material may fluoresce at more than one infrared wavelength. Those skilled in the art will readily be able to determine the suitable wavelength(s) at which to detect fluorescence for a particular material without undue experimentation.

In one embodiment of the invention, pre-cast sodium dodecylsulfate polyacrylamide gels are used, for example NuPAGE Novex 4-12% Bis-Tris gels commercially available from Invitrogen Corporation (Carlsbad, Calif., USA). Other sodium dodecylsulfate polyacrylamide gels may be suitable in the practice of this invention; those skilled in the art will readily be able to identify other suitable electrophoresis gels useful in the practice of the present invention without undue experimentation.

In one embodiment, prior to electrophoresis the sample to be analyzed is combined with a reducing agent, such as NuPAGE sample reducing agent (10×) (Invitrogen Cat. No. NP0004) which contains dithiothreitol, or the like. In one embodiment, prior to electrophoresis the sample to be analyzed is combined with a buffer NuPAGE LDS sample buffer (4×)—Invitrogen Cat. No. NP0007), which is 40-70% glycerol, or the like. In one embodiment, the reducing agent and buffer are combined with each other prior to combining with the sample to be analyzed. Those skilled in the art will readily be able to identify other suitable reducing agents and buffers useful in the practice of the present invention without undue experimentation.

In an embodiment of this invention in which a powdered infant formula is to be analyzed, the powder is dissolved in water to form a solution. In one embodiment, a liquid infant formula is diluted with water to form a solution to be analyzed.

In one embodiment, the invention of this invention includes: preparing standard solutions having different known concentrations of lactoferrin in the approximate range of 0.01-0.10 mg/ml and independently combining each standard solution with a sample reducing agent and a buffer to produce standard sample solutions having lactoferrin concentrations of approximately 0.005-0.075 mg/ml; preparing a solution of said infant formula and combine it with a sample reducing agent and a buffer to produce an infant formula sample solution; providing a sodium dodecylsulfate polyacrylamide gel for the standard sample solutions and infant formula sample solution; heating the sample solutions at about 95° C. for about 8 minutes; and loading each such sample solution into its respective unit or well of the gel; performing electrophoresis on the gel using substantially the same electrophoresis power level and time period for each sample solution; staining the gel with a Coomassie blue stain and then destaining, leaving on the gel only the stain that has bound to the lactoferrin; using an infrared fluorescence imaging system to quantitatively measure the infrared fluorescence of the stain on each unit of gel at a wavelength that is in the range of 680-700nm; and, comparing the measured infrared fluorescence of the stain on the unit of gel loaded with infant formula sample solution to the infrared fluorescence of the stain on the units loaded with the standard sample solutions to determine the lactoferrin concentration in the infant formula.

In one embodiment, the infant formula sample(s) and the standard solutions are all loaded onto the same gel, in separate wells, and all are analyzed at the same time.

Heat treatment of infant formula is standard practice to sterilize the formula, but it results in denaturing and aggregation of proteins in the formula, such as lactoferrin. To accurately measure the total amount of lactoferrin in a heat-treated infant formula sample using SDS-PAGE and infrared fluorescence detection, it is necessary to break up the aggregates by performing the SDS-PAGE under reducing conditions in which the sample to be analyzed is heated sufficiently to break up the aggregates. It has been discovered that heating for about 7-9 minutes at about 90-100° C. is suitable for obtaining an accurate analysis of total lactoferrin.

The following examples are presented to illustrate certain embodiments of the present invention, but should not be construed as limiting the scope of this invention.

### EXAMPLE 1

### Method for Analysis of Lactoferrin in Finished Product

### A. Equipment

Odyssey Infrared Imaging System (LI-COR)
Micro centrifuge—VWR Galaxy Mini Spin—Cat. No. 37000-700 or equivalent.
Orbital shaker—Labline Maxi-Rotor or equivalent
Vortex Mixer—VWR Cat. No. 58816 or equivalent.
Electrophoresis system:
Novex Mini-cell Xcell Surelock—Invitrogen Cat. No. E10001

### B. Materials

Pre-cast gels: NuPAGE Novex 4-12% Bis-Tris gel, 10 wells, 1.0 mm—Invitrogen Cat. No. NPO321 Box
Micro centrifuge tubes—1.5 ml (polypropylene)—VWR Cat. No. 20170 or equivalent Simply Blue Safe Stain (1×)—Invitrogen Cat. No. LC6060
Lactoferrin standard—Sigma product # 9507
Water—MilliQ water or deionized
NuPAGE MOPS SDS Running buffer (20×)—Invitrogen Cat. No. NP0001
Running buffer (1×)—mix 50 mL of NuPAGE MOPS SDS Running buffer (20×) and 950 mL of deionized water.
NuPAGE LDS sample buffer (4×)—Invitrogen Cat. No. NP0007.
NuPAGE sample reducing agent (10×)—Invitrogen Cat. No. NP0004.
Reducing sample buffer (1×)—1000 uL of NuPAGE LDS sample buffer with 400 uL of NuPAGE sample reducing agent (10×).

### C. Procedure

Stock Standard: Weigh about 24 mg of lactoferrin standard (about 87% pure), transfer to a 10 ml volumetric flask, add 8 ml water, sonicate for about 15 minutes to dissolve the LF and add water to volume; mix by vortex mixer. This lactoferrin stock solution is ˜2 mg/ml.

Working Standard: Make 1:10 dilution (add 100 ul of lactoferrin stock standard to 900 ul water in a micro centrifuge tube, mix by vortex). The final concentration of this lactoferrin working standard stock is ˜0.2 mg/ml.

Calibration Standards:
Calibration Standard 1: add 500.0 ul of lactoferrin working standard to 800.0 ul of water in a micro centrifuge tube and mix.
Calibration Standard 2: add 250.0 ul of lactoferrin working standard to 1050.0 ul of water in a micro centrifuge tube and mix.
Calibration Standard 3: add 125.0 ul of lactoferrin working standard to 1175.0 ul of water in a micro centrifuge tube and mix.
Aliquot 65 ul of each calibration standard into a micro centrifuge tube with a screw cap.
Store this calibration standard stock at −20° C.

Finished Product: Weigh about 20 mg of sample in a 5 ml volumetric flask, add about 4 ml water, sonicate for about 15 minutes to dissolve the sample, add water to volume, mix by vortex. Aliquot 65.0 uL sample each into 2 micro centrifuge tubes.

Prepare reduced standard and samples:
Add 35.0 ul of reducing sample buffer to each calibration standards and sample (65 ul). Mix by vortex. Heat the micro centrifuge tubes at 95° C. for 8 minutes. Let the tubes cool down for 1 minute. Spin down tubes in a Micro centrifuge for a few seconds.

Running SDS-PAGE: Rinse pre-cast gel with water. Insert the pre-cast gel into the Xcell Mini-cell apparatus. Fill the Xcell Mini-cell apparatus with running buffer (1X). Using gel loading pipette tips, load 20.0 uL of lactoferrin standards and samples into each well; all samples are duplicated. Cover Xcell Mini-cell apparatus and perform electrophoresis at 200V for 65 minutes., then place the gel in a staining dish. Wash the gel with water. Discard water. Replace it with approx. 20 mL of SimplyBlue™ SafeStain reagent. Shake for 1 hour. Discard staining reagent and replace it with water. Destain by leaving it over night (or 3 hours) with shaking at medium setting.

Gel scanning and Data Analysis: The gels are scanned with the Odyssey Infrared Imaging System. The lactoferrin calibration standards is used to make a standard curve (Integrated Intensity v. Concentration). The lactoferrin concentrations of the samples are determined by measuring their IR integrated intensity, and using the standard curve to determine the lactoferrin concentration which correlates with the measured intensity.

### EXAMPLE 2

### Validation of Method of Measuring Lactoferrin in Infant Formula

A bovine lactoferrin reference standard from Sigma (product # L9507, Lot # 097K3779, purity: 87.42% based on COA) was used to test the method.

Linearity was determined by using 4 different amounts of bovine lactoferrin reference standards over a range of approximately 1 ug to 0.125 ug on a the same day. A least-square regression equation was constructed using the standard amount versus the instrument response. The correlation coefficient was 0.998, and the method was linear over the entire range.

Repeatability was determined by measuring the lactoferrin content 6 independent samples of the same product on the same day. Each sample was measured twice on separate gels. The results are shown in Table 1, below.

TABLE 1
Data for Repeatability

| | Gel 1 | Gel 2 | AVG | STDEV | CV |
|---|---|---|---|---|---|
| | g/100 g | g/100 g | g/100 g | g/100 g | % |
| Sample 1 | 1.089 | 1.104 | 1.10 | 0.01 | 0.97 |
| Sample 2 | 1.105 | 1.103 | 1.10 | 0.00 | 0.13 |
| Sample 3 | 1.054 | 1.057 | 1.06 | 0.00 | 0.22 |
| Sample 4 | 1.047 | 1.071 | 1.06 | 0.02 | 1.63 |
| Sample 5 | 0.984 | 1.029 | 1.01 | 0.03 | 3.12 |
| Sample 6 | 0.926 | 0.937 | 0.93 | 0.01 | 0.84 |
| AVG | 1.03 | 1.05 | | | |
| STDEV | 0.07 | 0.06 | | | |
| CV % | 6.53 | 5.94 | | | |

Intermediate precision was determined by measuring 6 independent samples of the same product on three different days by two analysts. The results are shown in Tables 2 and 3, below.

TABLE 2
Data for Precision (Analyst 1)

| | Day 1 | Day 2 | Day 3 | 3 Days |
|---|---|---|---|---|
| | g/100 g | g/100 g | g/100 g | g/100 g |
| Sample 1 | 1.096 | 1.071 | 0.9975 | |
| Sample 2 | 1.104 | 1.020 | 0.9733 | |
| Sample 3 | 1.056 | 1.053 | 0.8831 | |
| Sample 4 | 1.059 | 1.021 | 0.9552 | |
| Sample 5 | 1.006 | 0.988 | 0.9583 | |
| Sample 6 | 0.931 | 0.925 | 0.9382 | |
| AVG | 1.04 | 1.01 | 0.95 | 1.00 |
| STDEV | 0.06 | 0.05 | 0.04 | 0.06 |
| CV % | 6.19 | 5.12 | 4.08 | 6.30 |

TABLE 3
Data for Precision (Analyst 2)

| | Day 1 | Day 2 | Day 3 | 3 Days |
|---|---|---|---|---|
| | g/100 g | g/100 g | g/100 g | g/100 g |
| Sample 1 | 0.982 | 1.189 | 1.018 | |
| Sample 2 | 0.970 | 1.099 | 1.004 | |
| Sample 3 | 0.945 | 1.186 | 0.846 | |
| Sample 4 | 1.036 | 1.210 | 1.106 | |
| Sample 5 | 0.910 | 1.092 | 1.078 | |
| Sample 6 | 0.902 | 1.042 | 1.058 | |
| AVG | 0.96 | 1.14 | 1.02 | 1.04 |
| STDEV | 0.05 | 0.07 | 0.09 | 0.10 |
| CV % | 5.20 | 5.95 | 9.10 | 9.85 |

Accuracy was determined by spiking commercially available Wyeth S26 Gold Infant Formula with 1.334 wt %, 1.068 wt % and 0.795 wt % of the reference standard, in duplicate. Both spiked samples were tested separately by two different analysts. Results are shown in Tables 4 and 5, below.

TABLE 4
Spike and Recovery (Analyst 1)

| | | LF | LF | | |
|---|---|---|---|---|---|
| | | Added | Found | | |
| spike % | | g/100 g | g/100 g | Recovery % | Ave % |
| 110% | LF1 | 1.334 | 1.411 | 105.7 | 104.4 |
| | LF2 | 1.334 | 1.374 | 103.0 | |
| 88% | LF1 | 1.068 | 1.128 | 105.7 | 105.3 |
| | LF2 | 1.068 | 1.120 | 105.0 | |
| 66% | LF1 | 0.795 | 0.768 | 96.6 | 101.8 |
| | LF2 | 0.795 | 0.850 | 106.9 | |

TABLE 5
Spike and Recovery (Analyst 2)

| | | | LF | | |
|---|---|---|---|---|---|
| | | LF Added | Found | | |
| spike % | | g/100 g | g/100 g | Recovery % | Ave % |
| 110% | LF1 | 1.334 | 1.368 | 102.5 | 105.7 |
| | LF2 | 1.334 | 1.454 | 109.0 | |
| 88% | LF1 | 1.068 | 1.094 | 102.5 | 111.1 |
| | LF2 | 1.068 | 1.278 | 119.7 | |
| 66% | LF1 | 0.795 | 0.962 | 121.1 | 117.4 |
| | LF2 | 0.795 | 0.903 | 113.7 | |

System suitability was determined by analyzing 6 loadings of the same sample. The CV % ranged from 2.277% to 5.320%

Ruggedness was evaluated by having three analysts analyze 6 samples that contained concentrations of lactoferrin unknown to the analysts, with the concentrations ranging from 1 g/L to 2 g/L lactoferrin. The lactoferrin determinations by the three analysts ranged from 85-119% of the actual amounts of lactoferrin in the samples.

These results indicated that the method has good linearity, repeatability, ruggedness, system suitability, precision and accuracy.

Many variations of the present invention not illustrated herein will occur to those skilled in the art. The present invention is not limited to the embodiments illustrated and described herein, but encompasses all the subject matter within the scope of the appended claims.

1. A method of quantitatively measuring the total lactoferrin content of an infant formula comprising the steps:
preparing a solution of the infant formula and combining it with a sample reducing agent to produce an infant formula sample solution;
loading the infant formula sample solution onto a sodium dodecylsulfate polyacrylamide gel to obtain a loaded gel;
heating the infant formula sample solution at about 90-100° C. for about 7-9 minutes;
performing electrophoresis on the loaded gel;
staining the gel with a Coomassie blue stain and then destaining the gel, leaving on the gel only the stain that has bound to the lactoferrin; and
quantitatively measuring the infrared fluorescence intensity of the stain on the gel at a suitable infrared wavelength.

2. The method of claim 1 comprising correlating the measured infrared fluorescence of the stain on the gel to an amount of lactoferrin using data derived from reference standards and then calculating the lactoferrin concentration of the infant formula by dividing the amount of lactoferrin so determined by the amount of infant formula loaded onto the gel.

3. The method of claim 2 wherein the data derived from reference standards is obtained by:
preparing solutions having a range known lactoferrin concentrations;
combining these solutions with a sample reducing agent to produce standard sample solutions;
loading each standard sample solution onto its respective unit of a sodium dodecylsulfate polyacrylamide gel to obtain a loaded gel;
heating the standard sample solutions at about 90-100° C. for about 7-9 minutes;
perform electrophoresis on the loaded gel using the procedure used for the infant formula;
staining the gels with a Coomassie blue stain and then destain, leaving on the gel only the stain that has bound to the lactoferrin; and,
quantitatively measuring the infrared fluorescence of the stain on each standard solution on the gel at the same fluorescence wavelength as used for the infant formula loaded gel.

4. The method of claim 1 wherein the infrared fluorescence intensity is measured at a wavelength of about 680-700 nm.

5. The method of claim 3 wherein the standard sample solutions and the infant formula sample solution comprise a buffer which is combined with the standard solutions and with the solution of infant formula before, after or simultaneously with the sample reducing agent.

6. The method of claim 5 wherein the buffer is mixed with the sample reducing agent prior to combining the buffer and sample reducing agent with the standard solutions and the solution of infant formula.

7. The method of claim 1 wherein the infant formula is in the form of a powder and the solution of infant formula is prepared by dissolving the powder in water.

8. A method of quantitatively measuring the total lactoferrin content of an infant formula comprising the steps of:
preparing standard solutions having different known concentrations of lactoferrin in the approximate range of 0.01-0.10 mg/ml and independently combine each standard solution with a sample reducing agent and a buffer to produce standard sample solutions having lactoferrin concentrations of approximately 0.005-0.075 mg/ml;
preparing a solution of the infant formula and combining it with a sample reducing agent and a buffer to produce an infant formula sample solution;
providing a sodium dodecylsulfate polyacrylamide gel for each of the standard sample solutions and infant formula sample solution and loading each such sample solution into its respective unit of the gel to obtain a loaded gel;
heating the infant formula sample solution and the standard sample solutions at about 95° C. for about 8 minutes;
performing electrophoresis on the loaded gel using substantially the same electrophoresis power level and time period for each sample solution on the gel;
staining the gel with a Coonnassie blue stain and then destain, leaving on the gel only the stain that has bound to the lactoferrin;
quantitatively measuring the infrared fluorescence of the stain on each unit of gel at a wavelength that is in the range of 680-700 nm with an infrared fluorescence imaging system; and
comparing the measured infrared fluorescence of the stain on the unit of gel loaded with infant formula sample solution to the infrared fluorescence of the stain on the units loaded with the standard sample solutions to determine the lactoferrin concentration in the infant formula.
